(19) United States
(12) Patent Application Publication
(10) Pub. No.: US 2005/0276823 A1
(43) Pub. Date: Dec. 15, 2005

(54) Methods and compositions for preventing oxidative degradation of proteins

(76) Inventors: John K. Cini, Blairstown, NJ (US); Athena D. Nagi, Framingham, MA (US)

Correspondence Address: LAHIVE & COCKFIELD, LLP., 28 STATE STREET, BOSTON, MA, 02109, US

(21) Appl. No.: 10/618,350

(22) Filed: Jul. 11, 2003

Related U.S. Application Data
(60) Provisional application No. 60/395,411, filed on Jul. 12, 2002.

(51) Int. Cl. A61K 9/00

(52) U.S. Cl. 424/400

(57) ABSTRACT

Methods and compositions for preventing oxidative damage to proteins, particularly antibodies, are provided. The compositions include a combination of metal chelators, such as DTPA, EGTA, and/or DEF, and can further include one or more free radical scavengers, particularly scavengers of oxygen radicals. Methods for enhancing protein stability using the compositions of the invention are also disclosed.

### RELATED APPLICATION

This application claims the benefit of U.S. Provisional Patent Application Ser. No. 60/395,411, filed Jul. 12, 2002, the entire contents of which application are incorporated herein by this reference.

### BACKGROUND OF THE INVENTION

Proteins undergo varying degrees of degradation during purification and storage. Oxidation is one of the major degradation pathways of proteins, and has a destructive effect on protein stability. Oxidative degradation of proteins results in the loss of electrons, which causes destruction of amino acid residues, protein aggregation [Davies, J. Biol. Chem. 262: 9895-901 (1987)], peptide bond hydrolysis [Kang and Kim, Mol. Cells 7: 553-58 (1997)], and hence protein instability due to alteration of the protein's tertiary structure.

Oxidation occurs via many different and interconnected pathways, and is catalyzed by a variety of triggering conditions, including elevated temperature, oxygen levels, hydrogen ion levels (pH), and exposure to transition metals, peroxides and light. Typically, a significant factor causing oxidative degradation of proteins is exposure to oxygen and metals. Certain excipients are formulated in pharmaceutical compositions to provide protection against aggregation, but can also enhance oxidation because they contain oxygen. For example, Tween contains trace amounts of peroxide contaminants, which can cause oxidation of the Tween in the presence of low concentration of metals. The combination of the oxygen radicals and metals results in the auto-oxidation and further breakdown of Tween, thereby providing a catalyst for the oxidation and, thus, degradation of the protein formulated with the Tween.

The advent of humanized and fully human antibodies for therapeutic use has created a need for maintaining protein stability in pharmaceutical compositions by preventing oxidative degradation. Oxidation of proteins such as monoclonal antibody-containing solutions can result in degradation, aggregation and fragmentation of the antibody, and thus loss of antibody activity. It is therefore desirable to formulate peptide- and antibody-containing pharmaceutical compositions with excipients that will protect proteins from oxidative damage due to a variety triggering factors. Thus, there is a need in the art to identify physical and chemical conditions that will remedy the acceleration of protein degradation, in order to provide stable protein-containing pharmaceutical compositions that can endure oxidative conditions over a period of time.

### SUMMARY OF THE INVENTION

The present invention provides improved compositions and formulations for protecting proteins against damage due to oxidation. The compositions contain one or more proteins susceptible to oxidation formulated together with a combination of metal chelators and, optionally, also one or more free radical scavengers, particularly scavengers of oxygen radicals (“ROS scavengers”). The compositions exhibit increased resistance from oxidation resulting in, for example, a longer product shelf life, greater stability allowing room temperature storage, and/or greater flexibility in product packaging. In addition, the compositions have been shown to exhibit a significant protective effect, even for multi-unit proteins which have one or more subunits or polypeptide chains and which are often particularly susceptible to oxidative damage. Accordingly, the present invention provides an important means for protecting (i.e., stabilizing) even multi-unit protein compositions, such as antibody compositions.

Accordingly, in one embodiment, the present invention provides a composition comprising a protein formulated (e.g., in a preparation, such as a laboratory-grade or pharmaceutical composition) with a combination of metal chelators selected from deferoxamine (DEF), diethylenetriamine pentaacetic acid (DTPA) and/or bis(aminoethyl)glycolether N,N,N′,N′-tetraacetic acid (EGTA). A preferred combination of chelators is DTPA and DEF which exhibit an unexpected synergistic effect in preventing against protein oxidation. Another preferred combination of chelators is EGTA and DEF.

Compositions of the present invention can further contain one or more agents which neutralize free radicals of oxygen (i.e., an ROS scavenger). Suitable ROS scavengers include, for example, mannitol, methionine and/or histidine. Accordingly, in another embodiment, the invention provides a composition containing one or more proteins formulated together with one or more metal chelators, such as DEF and/or DTPA, and one or more ROS scavengers, such as mannitol, methionine and/or histidine.

Any suitable protein or polypeptide of interest which is susceptible to oxidation can be protected and, thus, stabilized according to the present invention (i.e., can be formulated in an oxidation protected composition as described herein). The protein can be in its natural (e.g., native) form state or be modified by, for example, microencapsulation or conjugation. The protein can be therapeutic or diagnostic. Such proteins include, for example, immunoblobulins, bovine serum albumin (BSA), human growth hormone (hGH), parathyroid hormone (PTH) and adrenocorticotropic hormone (ACTH) against oxidative damage.

In addition, multi-unit proteins, such as antibodies, which are particularly susceptible to oxidative damage, protein aggregation and breakdown, rendering them diagnostically and therapeutically non-functional, can be protected according to the present invention. In a particular embodiment, the invention provides protected (i.e., stabilized) antibody compositions, such as those which include one or more monoclonal antibodies, including fully human antibodies, as well as fragments thereof and immunoconjugates (i.e., antibodies conjugated to therapeutic agents, e.g., as a toxin, a polymer, an imaging agent or a drug).

Compositions of the present invention can also include one or more agents which inhibit protein aggregation. In a particular embodiment, the agent is selected from polysorbate 80, polysorbate 20, glycerol and poloxamer polymers. The compositions can still further include a buffer that maintains the pH of the composition preferably from about 5.0 to about 8.0. Suitable buffers include, for example, Tris, acetate, MES, succinic acid, PIPES, Bis-Tris, MOPS, ACES, BES, TES, HEPES, EPPS, ethylenediamine, phosphoric acid, and maleic acid.

Accordingly, in another aspect, the present invention provides a method for preparing a stabilized protein composition by formulating a protein together with one or more metal chelators, ROS scavengers and/or other optional agents as described above.

Other features and advantages of the invention will be apparent from the following detailed description and examples which should not be construed as limiting. The contents of all references, patents and published patent applications cited throughout this application are expressly incorporated herein by reference.

### DETAILED DESCRIPTION OF THE INVENTION

The present invention provides methods and compositions for reducing or preventing oxidation of proteins which causes, for example, protein breakdown and aggregation. As shown herein, significant protection can be achieved by formulating proteins together with various combinations of oxidation protective compounds, such as transition metal chelators, ROS scavengers and other active agents. In a particular embodiment, the oxidation-protected compositions of the invention include monoclonal antibodies which are prone to damage by oxidative mechanisms and, therefore, difficult to maintain in stable form.

In particular, the present invention demonstrates for the first time that selected combinations of chelators, such as DEF combined with DTPA or EGTA, have a significant protective effect against protein oxidation caused by a variety of agents and environmental factors, such as metals (e.g., copper and iron), peroxides, temperature and light. The present invention further demonstrates the surprising result that DEF and DTPA exhibit a synergistic protective effect against oxidative degradation of proteins when used in combination (i.e., an effect greater than expected in comparison with the effect observed using either chelator alone). The invention further demonstrates that particular combinations of chelators and ROS scavengers, such as DTPA in combination with mannitol, methionine and/or histidine, provide a significant protective effect against protein oxidation.

In order that the present invention may be more readily understood, certain terms are first defined as set forth below. Additional definitions are set forth throughout the detailed description.

Definitions

As used herein, the following terms and phrases used to describe the invention shall have the meanings provided below.

The term “oxidation protective compound” refers to any substance that prevents, limits, reduces or otherwise controls the oxidation of a protein by, for example, chelating a metal which can cause or promote oxidation, or by scavenging free radicals of oxygen (referred to herein as “reactive oxygen species” or “ROS”). Oxidative protective compounds used in compositions of the invention generally provide a relative protection from oxidation of at least about 10%, preferably at least about 20%, more preferably at least about 40%, still more preferably at least about 60%, and most preferably at least about 80% or greater.

“Relative protection” as used herein, refers to the protection provided by one or more oxidation protective compounds compared to the oxidation which occurs in the absence of the one or more oxidation protective compounds. In a particular embodiment, relative protection (RP) is calculated as follows:
RP=100%−[(Intensity of a specific band in a sample treated with Asc and metals, containing protective compounds)÷(Intensity of a specific band in a sample treated with Asc and metals without protective compounds)]

Oxidation protective compounds of the present invention include, for example, transition metal chelators (e.g., DTPA, DEF, EGTA, etc.), ROS scavengers (e.g., mannitol, sorbitol, methionine, histidine, melatonin), and other agents which protect against protein oxidation.

The term “oxidation protected composition” refers to a composition containing one or more proteins susceptible to oxidation in combination with one or more oxidation protective compounds. Such compositions exhibit a decreased tendency toward oxidation, as shown by, for example, a reduction in the percentage of oxidation-related aggregates or degradants present. This can be measured by, for example, SDS-PAGE, or other biochemical or biophysical techniques, and quantified, for example, by determining the relative protection.

The term “neutralizes” refers to the capacity of one or more oxidation protective compounds, such as a chelator or ROS scavenger, to protect against oxidation, i.e., to act as an oxidation protective compound.

The terms “chelator”, “metal chelator”, “transition metal chelator” and other grammatical variations thereof, are used interchangeably and refer to a polyfunctional molecule which has a multiplicity of negatively charged and/or electron-rich ligands which can sequester metal ions with varying affinities. Suitable electron-rich functional groups include carboxylic acid groups, hydroxy groups and amino groups. Arrangement of these groups in aminopolycarboxylic acids, hydroxypolycarboxylic acids, hydroxyaminocarboxylic acids, and the like, result in moieties that have the capacity to bind metal, thereby removing it from solution and rendering it unavailable to react with O₂-containing compounds. Examples of chelators include aminopolycarboxylic acids, such as, ethylenediaminetetraacetic acid (EDTA), diethylenetriamine pentaacetic acid (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid; and hydroxyaminocarboxylic acids, such as, for example, N-hydroxyethyliminodiacetic acid (HIMDA), N,N-bis-hydroxyethylglycine (bicine) and N-(trishydroxymethylmethyl) glycine (tricine); and N-substituted glycines such as glycylglycine. Other candidate chelators include 2-(2-amino-2-oxoethyl) aminoethane sulfonic acid (BES) and deferoxamine (DEF). Suitable chelators used in a protein formulation of the present invention include, for example, those that bind to metal ions in solution to render them unable to react with available O₂, thereby minimizing or preventing generation of .OH radicals which are free to react with and degrade the protein. Such chelators can reduce or prevent degradation of a protein that is formulated without the protection of a chelating agent.

Chelating agents used in the invention can be present in their salt form e.g., carboxyl or other acidic functionalities of the foregoing chelators. Examples of such salts include salts formed with sodium, potassium, calcium, and other weakly bound metal ions. As is known in the art, the nature of the salt and the number of charges to be neutralized will depend on the number of carboxyl groups present and the pH at which the stabilizing chelator is supplied. As is also known in the art, chelating agents have varying strengths with which particular target ions are bound. In general, heavy metal ions are bound more strongly than their similarly charged lower molecular weight counterparts.

The terms “free radical oxygen scavengers”, “reactive oxygen species scavengers” and “ROS scavengers” are used interchangeably and refer to compounds that remove oxygen centered free radicals or ROS from solution. An oxygen centered free radical is any free radical with an oxygen center and two unpaired electrons in the outer shell. Free radicals are highly reactive due to the presence of unpaired electrons. The most common ROS include: the superoxide anion (O2-), the hydroxyl radical (.OH), singlet oxygen (1O₂), and hydrogen peroxide (H₂O₂). Suitable ROS scavengers of the invention include, but are not limited to, methionine, histidine and mannitol.

I. Factors Affecting Protein Stability

Oxygen/Oxidative Damage

Oxidation of proteins is one of the most common causes of degradation because it involves the participation of oxygen, a ubiquitous element. Reactive oxygen species, including hydrogen peroxide and the free super oxide (O₂—) and hydroxyl radicals (.OH), can cause considerable damage to proteins, including protein aggregation (Davies, J B C 1987 vol. 262 pg. 9895), peptide bond hydrolysis (Kang and Kim, Mol. Cells 1997 vol. 7 pg. 553) and intermolecular crosslinking dityrosines (Davies, J B C 1987 vol. 262 pg. 9908).

Typical purification and storage procedures can expose protein biotherapeutics to conditions and components that cause oxidative damage. Trace (ppm level) metals (Cu²⁺, Fe²⁺, Co²⁺ and Mn²⁺, iron and copper being most common (Packer, Method Enz. Vol 186 pg. 14) (Ahmed, J. Biol. Chem. 1975 vol. 250 pg. 8477) can leach out of final container packaging such as glass vials, promoting hydrolysis of the amide bond (Wang and Hanson, J. Parent. Sci. Tech. 1988 vol. 42 pg. s4-s25), enhancing oxidation and resulting in protein aggregation. Exposure to light can also create reactive species, participating in an oxidative cascade. Tween (polysorbate), a commonly used FDA approved surfactant, can contain reactive oxygen species as impurities (Packer, Method in Enzymology 1990 Vol. 186) that foster oxidative damage (Hunt, Biochem. J. 1988 vol. 250 pg. 87) (Chang and Bock, Anal. Biochem. 1980, vol. 104 pg. 112).

In addition, some antioxidants conventionally used to protect small molecules against oxidation, including, for example, thiol derivatives, sulfurous acid salts, such as sodium sulfate and ascorbic acid, are detrimental to proteins, especially large proteins such as monoclonal antibodies, since these additives are detrimental to disulfide bonds.

Accordingly, the present invention provides methods and compositions that reduce oxidative damage in protein formulations by controlling one or more of the aforementioned oxidative mechanisms. This can result in, for example, improved product stability and/or greater flexibility in manufacturing processes and storage conditions.

Temperature and pH

Most protein chemical degradative processes are temperature dependent. In the case of oxidation, however, paradoxes exist. Lower temperatures increase oxygen solubility, but decrease rates of oxidative degradation; higher temperatures decrease oxygen solubility while increasing rates of oxidative degradation (J. Par. Sci Tech. Vol. 36, 1982, pg. 222).

pH is another factor that influences oxidation. As pH is increased above 7.0, hydrogen ion concentration increases, and with it, so does the oxidation potential (Nernst equation). The effects of pH on peptide hydrolysis are well documented, and can occur at both acid and alkaline pHs in MAbs. Protein hydrolysis can occur under acidic conditions at site with amino acids sequences: X-Asp-X, Ser/Thr-X, Pro-X or under alkaline conditions at X-Asn-X, X-Asp-X (Volkin, Mol. BioTech. 1997 vol. 8, pg. 105) (Reubsaet, J. Pharm. BioMed. Anal. 1998 vol. 17, pg. 955). Ser-X and Thr-X cleaved under acidic conditions are affected by the microenvironment and adjacent amino acid on the N or C terminal side (Wang and Hanson, J. Parent. Sci. Tech. 1988 vol. 42 pg. s4-s25). Pro-X under acidic and oxidative conditions forms glutamyl semialdehyde or is hydrolyzed at 2-pyrolidone and a new N-terminal is formed (Reubsaet, J. Pharm. Biomed. Ana. 1998 vol. 17 pg. 955).

II. Forms of Non-Reducible Covalent Protein Aggregates

Most protein aggregate forms are the result of new inter-disulfide cross-linkages or newly formed non-disulfide cross-linkages. Two types of non-reducible cross linkages caused by oxidation involve Trp conversion to a kynurenine (an open pentyl ring structure) resulting in a decrease in fluorescence emission and protein aggregation, or the formation of inter-dityrosines resulting in fluorescence spectra emission increase at 410-420 nm (excitation at 315 nm) (Wold and Moldave, M E, 1984 vol. 107, pg. 377).

Other forms of non-reducible disulfide cross linkages are amidation (Lys amide plus carboxyl groups under acid conditions) or transamidation (Lys amide+Asn/Gln under acid or alkaline conditions). Transamidation of proteins can be enhanced/accelerated in the presence of metals (Hirs and Timasheff, M E. 1972 vol. 25, pg. 411).

Beta elimination is the reduction of disulfide bonds, formation of persulfide, thioaldehyde to aldehyde, and the formation of a reactive dehydroalanine that is accelerated at alkaline pH. The dehydroalanine can form new non-reducible cross linkage with Tyr, Lys, His, Arg and Cysteine, and under acidic conditions peptide hydrolysis occurs on the C terminal side of dehydroalanine (“Chemical Deterioration of Protein” 1980 Whitaker J. ACS Symp Ser. 123 Pg. 147).

III. Analytical Techniques For Determining Protein Degradation Levels

As described herein, the present invention uses, in one embodiment, a validated method for determining the levels of protein degradation by chemical compounds. This method can be used to identify compounds that protect against such degradation, as well as to determine the level of protection provided. In particular, the provided method involves enhancing oxidative damage of a protein, and confirming that this damage generates the same species observed during real-time and accelerated aging. In a particular embodiment, oxidative conditions are simulated by exposing samples to sodium ascorbate (e.g., 4 mM, pH 7.5, 37° C., for 48 hours). Oxidative species can be visualized by running samples on SDS-PAGE, followed by silver-staining. For our analyses, 3 μg of material was loaded—a higher than typical load amount for silver-staining, which has nanogram level sensitivity. This guarantees detection of species even at relatively low abundances. Densitometry methods are known in the art to analyze gel band intensities for straightforward comparisons between samples. This method can be used, for example, to assess the level of protection provided by oxidation productive compounds in the context of various metals. A number of analytical methods such as RP-HPLC, UV-measurements, fluorescence measurements and isocratic elution can be used to confirm the presence of oxidized products (Reubasaet et al., (1998) J. Pharm. Biomed. Anal. 17: 955-978). Proper oxidation is typically accomplished by exposing samples to sodium ascorbate at pH 7.5, 37° C., for 48 hours. Oxidation can be confirmed by analytical methods such as visualization by silver-stained SDS-PAGE.

Other recognized methods of enhancing oxidative damage corresponding to damage that occurs during real-time and accelerated aging are also encompassed by the present invention. For example, a number of oxidants such as alkaline media, copper, iron, peroxidase and ascorbic acid can be used.

IV. Proteins

Any protein susceptible to oxidation, including binding proteins, immunoglobins, enzymes, receptors, hormones and fragments thereof, can be stabilized (i.e., protected) by the methods and compositions of the present invention. The source or manner in which the protein is obtained or produced is of no consequence, e.g., whether isolated from cells or tissue sources by an appropriate purification scheme, produced by recombinant DNA techniques, or synthesized chemically using standard peptide synthesis techniques. For example, a wide variety of native, synthetic and/or recombinant proteins, including chimeric and/or fusion proteins, can be stabilized by the methods and compositions of the invention.

In a particular embodiment, the invention pertains to compositions and methods for stabilizing antibodies, including monoclonal antibodies and human antibodies. The terms “antibody” and “immunoglobin” are used interchangeably herein and include fragments and derivatives thereof.

An antibody used in the present invention can be polyclonal or monoclonal. The term “monoclonal antibody” or “monoclonal antibody composition”, as used herein, refers to a population of antibody molecules that contain only one species of an antigen binding site capable of immunoreacting with a particular epitope. The invention also pertains to recombinant antibodies stabilized by the compositions and methods of the invention. Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine mAb and a human immunoglobulin constant region. Single-chain antibodies have an antigen binding site and consist of a single polypeptide. Multi-specific antibodies are antibody molecules having at least two antigen-binding sites that specifically bind different antigens. Such molecules can be produced by techniques known in the art. Humanized antibodies are antibody molecules from non-human species having one or more complementarity determining regions (CDRs) from the non-human species and a framework region from a human immunoglobulin molecule. Furthermore, fully human antibodies can be stabilized by formulations and methods of the invention, whether the antibody is derived from a human being or transgenic animal containing human genes.

Those of ordinary skill in the art will appreciate an antibody formulated using compositions and methods of the present invention can be fragments of antibodies, particularly fragments that contain an antigen-binding portion of an antibody. The term “antigen-binding portion” refers to one or more fragments of an antibody that retain the ability to bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., 1989, Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., 1988, Science 242:423-426; and Huston et al., 1988, Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

Accordingly, the present invention also provides stabilized therapeutic and/or diagnostic antibody compositions formulated as described herein. Suitable therapeutic antibodies include any antibody or fragment thereof, as well as antibody derivatives and immunoconjugates (e.g., antibody conjugated to a therapeutic moiety such as a cytotoxin, a therapeutic agent or a radioactive metal ion). A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Techniques for conjugating such therapeutic moiety to antibodies are well known in the art.

The invention also provides kits which include one or more proteins stabilized by (e.g., formulated in) an oxidation protective composition of the present invention and, optionally, instructions for use.

V. Oxidation-Protective Compounds

Metal chelators have been shown to inhibit/reduce free radical formation and Tween (polysorbate) oxidation. Their effectiveness, which varies depending on the experimental conditions, has been documented. The most commonly used chelator, EDTA, has been shown to inhibit the formation of free radicals in a Cu catalyzed Fenton reaction. In some cases, EDTA enhances free radical formation in a Fe catalyzed Fenton reaction (Bioch. Biophy. Acta 1997, vol. 1337, pg. 319). This occurs because the Fe-EDTA complex has an open structure allowing the hydroxyl radical (HO.) to escape. It has also been suggested that EDTA maintains the Fe in solution, preventing it from precipitating at physiological pH (ME 1990, vol 186 pg 16).

DTPA can reduce iron dependent hydroxyl radical (HO.) formation from O₂ and H₂O₂ (Packer, M E 1990, vol 186 pg. 42). DEF is a powerful inhibitor of iron-dependent lipid peroxidation (Packer, M E 1990, vol 186 pg. 42). Although both chelators have been used in protein formulations, they have never before been used together, such as in a formulation for protecting proteins against oxidation.

Other oxidative protective compounds of the invention include art recognized free radical scavengers including, for example, mannitol, an FDA approved excipient & OH. scavenger (Kocha, B B A vol. 1337, 1997 pg. 319), histidine (Kammeyer, B B A vol. 49, 1999 pg. 117), and melatonin (free radical scavenger) (Reiter, Nutr. 1998 vol. 19 pg. 691).

Oxidative protective compounds of the invention can also be combined with agents that prevent protein aggregation. This helps further prevent against damage and inactivation of protein samples and preparations. Suitable agents include, for example, a polysorbate (e.g., polysorbate 80 and/or polysorbate 20), a glycerol, a poloxamer polymer (e.g., poloxamer 407 and poloxamer 188), a polyethylene glycol, polyvinyl pyrrolidone, and Brij. Such agents are commercially available and well known in the art.

VI. Pharmaceutical Compositions

Oxidation protective compounds of the invention can be incorporated into pharmaceutical compositions suitable for administration. Oxidative protective compounds of the invention also can be incorporated into compositions suitable for diagnostic and/or laboratory purposes. Such compositions typically include the protein of interest, along with a combination of oxidation protective compounds and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Accordingly, the present invention further provides diagnostic and therapeutic pharmaceutical compositions containing stabilized proteins, as well as methods for preparing such compositions by formulating the proteins together with a combination of oxidation protective compounds and a pharmaceutically acceptable carrier. Such compositions can further include additional agents, including polysorbates and glycerol, at varying concentrations, and various buffers that maintain the pH from, for example, about 5.0 to about 8.0. Thus, in a particular embodiment, the invention provides a method for preparing an oxidation protected composition by formulating one or more proteins together with DEF and EGTA or DTPA, optionally in combination with an ROS scavenger, and a pharmaceutically acceptable carrier.

It is understood that appropriate doses of pharmaceutical compositions depends upon a number of factors within the knowledge of the ordinarily skilled physician, veterinarian, or researcher. The dose(s) will vary, for example, depending upon the identity, size, and condition of the subject or sample being treated, further depending upon the route by which the composition is to be administered, if applicable, and the effect which the practitioner desires the agent to have upon the protein composition formulated according to the invention. When one or more of these compositions is to be administered to an animal (e.g. a human) in order to modulate expression or activity of a protein of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium, and then incorporating the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches, and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes having monoclonal antibodies incorporated therein or thereon) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the colon epithelium). A method for lipidation of antibodies is described by Cruikshank et al. (1997) J. Acquired Immune Deficiency Syndromes and Human Retrovirology 14:193.

VII. Formulation of Exemplary Compositions

This section provides ranges and formulations for making exemplary compositions in accordance with the present invention. A skilled practitioner readily can formulate further alternative compositions within the scope of the invention using using no more than routine experimentation.

Suitable compositions can include one or more proteins at a concentration of from about 1 μg/mL to about 500 mg/mL, from about 50 μg/mL to about 300 mg/mL, or from about 1 mg/mL to about 100 mg/mL.

One or more metal chelators can be included at concentrations within the following exemplary ranges. Suitable compositions can include DTPA and/or EGTA at a concentration of from about 1 μM to about 10 mM, from about 10 μM to about 10 mM, from about 50 μM to about 5 mM, or from about 75 μM to about 2.5 mM. Additionally or alternatively, DEF can be included at a concentration of from about 1 μM to about 5 mM, from about 10 μM to about 1 mM, or from about 20 μM to about 250 μM.

Compositions containing one or more ROS scavengers can be formulated to include ROS scavengers at concentrations in the following exemplary ranges. Suitable compositions can include mannitol at a concentration of from about 0.01% to about 25%, 0.1% to about 25%, from about 0.5% to about 12%, or from about 1% to about 5%. Additionally or alternatively, the composition can include methionine at a concentration of from about 10 μM to about 200 mM, from about 100 μM to about 200 mM, from about 500 μM to about 100 mM, or from about 15 mM to about 35 mM. Additionally or alternatively, suitable compositions can include histidine at a concentration of from about 10 μM to about 200 mM, from about 100 μM to about 200 mM, from about 500 μM to about 100 mM, or from about 15 mM to about 35 mM.

Suitable compositions can include one or more polysorbates (e.g., polysorbate 80 and/or polysorbate 20) at a concentration of from about 0.0005% to 12%, from about 0.001% to about 0.1%, or from about 0.005% to about 0.1%. Additionally or alternatively, suitable compositions can include glycerol at a concentration of from about 0.1% to about 20%, or from about 1% to about 5%. Additionally or alternatively, suitable compositions can include one or more poloxamers (e.g., poloxamer 407 and/or poloxamer 188) at a concentration of from about 0.001% to about 30%, or from about 0.2% to about 10%.

Suitable compositions optionally can include a buffer to maintain the pH from about 5.0 to about 8.0, or from about 5.5 to about 7.5. The concentration of buffer can be from about 5 mM to about 100 mM, or from about 20 mM to about 50 mM.

One exemplary composition includes: a binding protein; from about 50 μM to about 5 mM DTPA; from about 10 μM to about 1 mM DEF; a buffer which maintains the pH of the composition from about 5.0 to 8.0; and one or more of the following agents: from about 0.0005% to about 12% polysorbate 20, from about 0.0005% to about 12% polysorbate 80, from about 0.1% to about 20% glycerol, from about 0.001% to about 30% polaxamer 407, and from about 0.001% to about 30% polaxamer 188. This composition also can include additional agents including methionine, histidine and/or mannitol.

In another exemplary composition, the composition includes: a binding protein; from about 50 μM to about 5 mM DTPA; one or more of the following agents: from about 0.5% to about 12% mannitol, from about 500 μM to about 100 mM histidine, and from about 500 μM to about 100 mM methionine; one or more of the following agents: from about 0.0005% to about 12% polysorbate 20, from about 0.0005% to about 12% polysorbate 80, from about 0.1% to about 20% glycerol, from about 0.001% to about 30% polaxamer 407, and from about 0.001% to about 30% polaxamer 188; and a buffer which maintains the pH of the composition from about 5.0 to 8.0.

Yet another exemplary composition includes DTPA, mannitol, a polysorbate, Tris, sodium chloride, and an antibody or an antibody fragment.

### EXAMPLES

Materials and Methods

1. Protein Oxidation Assay

Numerous methods of exposing proteins to oxidation are known in the art and can be used to test formulations for their ability to protect against oxidative conditions according to the present invention. Commonly used methods include exposure of proteins to H₂O₂ or ascorbate+metals and O₂ (exposure to air) (Reubsaet, J. Pharm. Biomed. Ana. (1998) 17: 955. In the present Example, oxidative conditions were simulated by treating samples with sodium ascorbate (4 mM concentration), 1 uM copper or iron at a pH of 7.5, with incubation at 37° C., typically for 48 hours. Except where noted, the sample protein concentration used was 1 mg/mL.

2. Accelerated Stability Studies

Selecting appropriate temperatures is important for accurate accelerated stability studies. It is well known in the art that elevated temperatures can result in irreversible denaturation of proteins occurring from partially unfolded structures. Such changes can complicate stability studies because chemical modifications observed in accelerated stability studies performed at temperatures at or above the start of a protein unfolding transition may not represent actual denaturation that occurs under typical storage conditions for final vialed product, e.g., at 4° C. Biophysical techniques such as calorimetry and fluorescence spectroscopy can be used to confirm whether elevated temperatures introduce such changes. Accordingly, for this study, 37° C. was found to cause no unfolding in protein structure.

3. Metal Chelators, Copper and Iron Solutions

To study the protective effect of metal chelators on proteins against copper and iron-mediated oxidative damage, metal chelators, such as EDTA, EGTA, DTPA, and DEF were investigated in a series of studies performed under accelerated oxidizing conditions as described in section 1 above. Protein samples were analyzed by SDS-PAGE, GPC-HPLC, and ELISA (selected samples).

4. SDS-PAGE

SDS-PAGE was performed using Bio-Rad Criterion gels (4-20% for reduced samples, 4-15% for nonreduced samples). All gels were loaded at a constant load amount of 3 μg sample per well. GPC-HPLC analysis was performed with 75 μg injections using a Tosohaas TSK3000 SWXL column (7.8 mm×30 cm).

5. Bioactivity Studies

Bioactivity was determined using an ELISA specific for an anti-T lymphocyte antigen antibody. Ninety six well plates were coated with a soluble T lymphocyte antigen. The antibody was added at various concentrations to the plates and allowed to bind soluble T lymphocyte antigen. Bound antibody was detected with anti-human IgG alkaline phosphatase conjugated antibody followed by the phosphatase substrate para nitrophenyl phosphate. The OD₄₀₅ was measured using an ELISA plate reader. Activity reported is relative to 100% binding activity of a reference standard of anti-T lymphocyte antigen antibody that was exposured to neither oxidants nor oxidation protective compounds.

6. Assessing Effectiveness of Oxidation Protection

Protein samples were incubated for 48 hours at elevated temperature (37° C.), but lower than the transition temperature necessary for protein unfolding. To analyze the degree of protection from oxidation, the protein samples were run on a reducing SDS-PAGE and visualized using silver-staining. The intensity of the protein bands was quantitated using densitometry. The intensity of oxidation-related species in a control, composition was compared with the intensity of the oxidation-protected composition.

The ranges of protection were categorized as follows:
10% reduction in band intensity or greater, or at least about 10% relative protection;
20% reduction in band intensity or greater, or at least about 20% relative protection;
40% reduction in band intensity or greater, or at least about 40% relative protection;
60% reduction in band intensity or greater, or at least about 60% relative protection;
80% reduction in band intensity or greater, or at least about 80% relative protection.

### Example 1

### Effect of Chelators on Copper and Ascorbate Induced Oxidation of Proteins

Chelators were added to ascorbate treated samples containing monoclonal antibodies with and without added copper, and degradation was evaluated at three time points (48, 96, 144 hours). In addition, samples containing Tween-80 (with and without DTPA) were also evaluated. The results are shown in Table 1.

TABLE 1

| Sample | Description | Damage |
|---|---|---|
| 1 | Ref. Standard (anti-T | Few distinct bands |
| | lymphocyte antigen | |
| | antibody) without | |
| | additives to induce | |
| | oxidation, and without | |
| | oxidation protective | |
| | compounds | |
| 2 | +4 mM Ascorbate | Slight increase in aggregates |
| | | and breakdown products |
| 3 | +Asc + 0.1 mM EDTA | Considerable increase in aggregates |
| | | and breakdown |
| 4 | +Asc + 1.0 mM EDTA | Comparable to sample 3 |
| 5 | +Asc + 0.1 mM DTPA | Comparable to sample 1 |
| 6 | +Asc + 1.0 mM DTPA | Comparable to sample 1 |
| 7 | +Asc + 0.1 mM DEF | Slight increase in one aggregate |
| | | species and one heavy-chain |
| | | fragment, excellent protection |
| | | with breakdown |
| 8 | +Asc + 1.0 mM DEF | Comparable to sample 7 |
| 9 | +1 μM Cu | Slight increase in one |
| | | heavy-chain fragment |
| 10 | +Cu + Asc | Much aggregation and breakdown |
| 11 | +Cu + Asc + 0.1 mM | Much aggregation and breakdown |
| | EDTA | |
| 12 | +Cu + Asc + 1.0 mM | Much aggregation and breakdown |
| | EDTA | |
| 13 | + Cu + Asc + 0.1 mM | Excellent protection, comparable |
| | DTPA | to sample 1 |
| 14 | +Cu + Asc + 1.0 mM | Excellent protection, comparable |
| | DTPA | to sample 1 |
| 15 | +Cu + Asc + 0.1 mM | Much aggregation and breakdown |
| | DEF | |
| 16 | +Cu + Asc + 1.0 mM | Much aggregation and breakdown |
| | DEF | |
| 17 | +Cu + Asc + 0.02% | Much aggregation and breakdown |
| | Tween-80 | |
| 18 | +Cu + Asc + 1.0 mM | Comparable to sample 1 |
| | DTPA + Tween-80 | |

Results:

Ascorbate treatment of samples containing anti-T lymphocyte antigen antibody at 37° C. for 48 hours clearly enhanced degradation and aggregation as visualized by silver-stained SDS-PAGE gel. New bands were observed, and specific aggregates and breakdown products commonly associated with the monoclonal antibodies at low levels (i.e., present in the reference standard at less than 5% intensity of total intensity of the bands) were enhanced upon ascorbate treatment. The oxidative damage was worsened by the presence of copper ion, with formation of approximately 12 bands, representing both aggregates and breakdown products. The addition of EDTA (0.1 or 1 mM) enhanced oxidative damage.

In contrast, DTPA (0.1 and 1 mM) had a strong protective effect (i.e., decreased oxidation as evidenced by a reduction in aggregate and breakdown products of the antibody) in both the absence and presence of copper. While DEF provided some protection against oxidative damage when no copper was present, damage was observed in the samples containing DEF copper that were treated with DEF. The addition of 1 mM DTPA to a solution containing the anti-T lymphocyte antigen antibody and 0.02% Tween-80 was also protective, greatly reducing the number and intensity of the bands which reflect much oxidative damage.

As the incubation time was increased, the extent of oxidative damage also increased, which was visualized by SDS-PAGE as an increase in band intensity of up to tenfold for existing polypeptide breakdown products, and formation of new bands for new polypeptide breakdown products. For example, at 144 hours incubation, unprotected samples experienced heavy oxidation, which appeared as a dark smeary band on the SDS-PAGE. But, importantly, even after a longer incubation time, the protective effect of DEF and DTPA was apparent, as visualized on SDS-PAGE by minimal increase in band intensity and minimal formation of new bands.

There was no apparent difference in the intensity of bands for samples treated with 0.1 vs. 1.0 mM DTPA, suggesting that maximum protection was achieved at the lower concentration. Thus, lower chelator concentrations were tested to identify a minimum concentration for protection.

Since ascorbate treatment is relatively harsh, generating oxidative species, an additional experiment was run to examine protein species generated by elevated temperature rather than the addition of ascorbate and metal. Samples of anti-T lymphocyte antigen antibody that were incubated at higher temperatures (45 and 53° C.) for several weeks without ascorbate and metal were run on an SDS-PAGE gel with samples that had been exposed to ascorbate and metal. The oxidative species visualized (as bands on SDS-PAGE) from the ascorbate-treated lanes were the same (based on alignment of bands on SDS-PAGE) as the non-ascorbate treated lanes under both reducing and non-reducing conditions. This demonstrates the validity of the ascorbate treatment protocol for generating relevant aggregation and breakdown species of the antibody.

### Example 2

### Effect of Chelators, Tween-80 and Protein Concentration on Metal and Ascorbate Induced Oxidation of Proteins

The following experiment focused on two main parameters: effective chelator concentrations (e.g., examining concentrations lower than those used in Example 1) and differential effects based on varying metal treatment.

Samples containing monoclonal antibody against T lymphocyte antigen and 0.02% Tween-80 were treated with 0.025, 0.05, 0.075, and 0.1 mM of chelator and incubated for 48 hours at 37° C. The samples were additionally treated with either copper or iron or no metals. Also, an additional protein sample contained a higher (5 mg/mL) protein concentration during its exposure to copper and ascorbate treatment. The results are shown in Table 2.

TABLE 2

| Sample | No Metal | Copper | Iron |
|---|---|---|---|
| Control | | None | None |
| Ascorbate | Up to 10× increase | Substantial (up to | Substantial (>20×) |
| | in aggregation and | 16×) increase in | increase in |
| | breakdown | aggregation and | aggregation and |
| | | breakdown | breakdown |
| Ascorbate + EDTA | Up to 18× increase | Substantial increase in | Extreme amount |
| | in aggregation and | aggregation and | (>100×) of |
| | breakdown | breakdown | aggregation and |
| | | | breakdown |
| +Asc + 0.025 mM | Excellent protection - | Excellent protection - | Substantial (up to |
| DTPA | comparable to | comparable to control | 10×) increase in |
| | control (RP 60% | (RP 80% and higher) | aggregation and |
| | and higher) | | breakdown |
| +Asc + 0.05 mM | Excellent protection - | Excellent protection - | Substantial (up to |
| DTPA | comparable to | comparable to control | 10×) increase in |
| | control (RP 60% | (RP 80% and higher) | aggregation and |
| | and higher) | | breakdown |
| +Asc + 0.075 mM | Excellent protection - | Excellent protection - | Substantial (up to |
| DTPA | comparable to | comparable to control | 10×) increase in |
| | control (RP 60% | (RP 80% and higher) | aggregation and |
| | and higher) | | breakdown |
| +Asc + 0.1 mM | Excellent protection - | Excellent protection - | Substantial (up to |
| DTPA | comparable to | comparable to control | 10×) increase in |
| | control (RP 60% | (RP 80% and higher) | aggregation and |
| | and higher) | | breakdown |
| +Asc + 0.025 mM | Some (10×) | Extreme amount of | Some increase in |
| DEF | increase in one | aggregation and | one aggregate, one |
| | aggregate, one | breakdown | heavy-chain related |
| | heavy-chain related | | fragment, protective |
| | fragment, | | against breakdown |
| | protective against | | (RP 40% and higher) |
| | breakdown (RP | | |
| | 60% and higher) | | |
| +Asc + 0.05 mM | Some (10×) | Extreme amount of | Some increase in |
| DEF | increase in one | aggregation and | one aggregate, one |
| | aggregate, one | breakdown | heavy-chain related |
| | heavy-chain related | | fragment, protective |
| | fragment, | | against breakdown |
| | protective against | | (RP 40% and higher) |
| | breakdown (RP | | |
| | 60% and higher) | | |
| +Asc + 0.075 mM | Some (10×) | Extreme amount of | Some increase in |
| DEF | increase in one | aggregation and | one aggregate, one |
| | aggregate, one | breakdown | heavy-chain related |
| | heavy-chain related | | fragment, protective |
| | fragment, | | against breakdown |
| | protective against | | (RP 40% and higher) |
| | breakdown (RP | | |
| | 60% and higher) | | |
| +Asc + 0.1 mM | Some (10×) | Extreme amount of | Some increase in |
| DEF | increase in one | aggregation and | one aggregate, one |
| | aggregate, one | breakdown | heavy-chain related |
| | heavy-chain related | | fragment, protective |
| | fragment, | | against breakdown |
| | protective against | | (RP 40% and higher) |
| | breakdown (RP | | |
| | 60% and higher) | | |
| 5 mg/mL | (Not tested) | Excellent protection - | (Not tested) |
| sample, +Asc + 0.1 mM | | comparable to control | |
| DTPA | | | |

Results:

In the absence of metal, DTPA exhibited a protective effect, even at 0.025 mM, while DEF treated samples were protected to some extent, as visualized by silver-stained SDS-PAGE.

In the presence of copper, the same trend observed in Example 1 (SDS-PAGE) was observed, i.e., DTPA protects the antibody, even at concentrations as low as 0.025 mM, while DEF enhanced the destructive oxidation.

In the presence of iron, samples having DTPA exhibited enhanced oxidative damage, while DEF samples showed protection similar to DEF samples without metal added, demonstrating the usefulness of DEF in protecting proteins from iron-mediated oxidative damage.

These data clearly show a metal-dependent difference in the oxidative protective effect afforded by different metal chelators. DTPA protects better against copper, whereas DEF protects against iron. Furthermore, higher antibody concentration does not appear to affect the protective effect of DTPA, since the same pattern of bands was observed on SDS-PAGE for a sample containing 5 mg/mL antibody as a 1 mg/mL sample.

### Example 3

### Synergistic Effect of a Combination of DTPA and DEF on Protection from Protein Oxidation

The foregoing study (Example 2) showed that DTPA and DEF have a metal-specific protective effect against oxidation. Specifically, DTPA has a protective effect against copper mediated protein damage and DEF has a protective effect against iron-mediated damage. Since copper and iron are both commonly found in pharmaceutical grade glass, the effect of combinations of DTPA and DEF treated with copper and iron, together and separately, were studied. In addition, whether higher concentrations of DTPA (greater than 0.1 mM) protects against oxidation in the presence of iron was also studied. Therefore, protein samples were evaluated containing monoclonal antibody, 0.02% Tween-80, copper or iron or both metals, and varying concentrations of DTPA concentrations or DTPA/DEF combinations.

Results:

At higher concentrations of DTPA (0.5 and 1.0 mM), there was a protective effect seen in samples treated with iron. There was a striking protective effect by a combination of DTPA and DEF seen in samples treated with copper or iron or both metals. Specifically, as shown in Examples 1 and 2, protein samples treated with either 0.1 mM DTPA or DEF showed some oxidative damage by different patterning of bands on SDS-PAGE. However, a combination treatment of 0.1 mM DTPA and DEF had a much greater protective effect than was expected in comparison with the observed protection from the individual chelators. Furthermore, a combination of 1 mM DTPA and 0.1 mM DEF provided less protection than the 0.1 mM DTPA/DEF combination.

In addition to the anti-T lymphocyte antigen antibody sample examined in Examples 1 and 2, this synergistic protective effect of DTPA and DEF against protein oxidation was also observed in other protein samples investigated. For example, the degradation products were greater for certain antibodies. For certain antibodies, two or more oxidation protective compounds restored band intensity back down to levels seen with samples not treated with ascorbate and metals, while for other antibodies, the combination lessened the intensity but showed less than complete protection. The unexpected synergistic effect between DTPA and DEF was explored further in Example 4.

### Example 4

### Effect of Chelator Concentration on Synergism between DTPA and DEF in Protection against Protein Oxidation

The synergistic protective effect of DTPA and DEF against oxidation of protein formulations was explored by formulating protein compositions with varying concentrations of DTPA and DEF. Specifically, protein samples containing antibody, 0.02% Tween-80 were exposed to copper and iron together and treated with different concentrations of DTPA/DEF.

Results:

All samples tested showed some degree of protection of antibodies from oxidative damage. Some combinations helped prevent higher molecular weight aggregation, but did not prevent species formation, which elute between the heavy and the light chain bands. The greatest protection was observed in samples treated with 1 mM DTPA/0.5 mM DEF, 0.02 mM DTPA/0.1 mM DEF, and 0.1 mM DTPA/0.5 mM DEF.

After silver-staining, the SDS-PAGE gels were scanned using a BIO-RAD GS-800 densitometer. Densitometric analysis provides an “adjusted volume” (i.e., the intensity of a band integrated over its volume and adjusted for any staining background). The intensity of bands was compared using Quantity One software to quantify the protective effect, These bands represent specific oxidative species, including aggregates and antibody breakdown products, that were consistently observed throughout the experiments.

Based on the adjusted volumes from densitometric data of representative band/oxidative species, it was apparent that the relationship between protection from oxidation and DTPA/DEF concentration is not simply additive. Combinations of DTPA and DEF that were effective for minimizing one species did not necessarily prevent the formation of another species. The specific concentration ranges that were most effective (i.e., that minimized oxidative bands) are DTPA concentrations of 0.1 to 0.5 mM with DEF concentrations of 0.02 to 0.1 mM. These results demonstrate that even at minimal chelator concentration (e.g., 0.02 mM DEF/0.1 mM DTPA) significant protection is afforded by the combination of DTPA and DEF.

### Example 5

### Effect of DTPA, DEF and EGTA on Oxidative Damage Enhanced by EDTA

Observation of a synergistic effect between DTPA and DEF led to studies to determine whether either or both chelators could “rescue” the oxidative damage enhanced by EDTA. In addition, the ability of EGTA to provide a protective effect alone or in combination with the previously examined chelators was studied. Specifically, protein samples containing 0.02% Tween-80 and EGTA or EDTA in combination with other chelators were exposed to copper and/or iron and evaluated.

Results:

In the presence of DTPA, DEF, or both chelators, severe oxidative damage was observed in samples treated with EDTA. One mM EGTA had considerable protection against Cu-based oxidation (comparable with control), with lesser protection against oxidation caused by Fe (as evidenced by additional aggregates and breakdown products compared with control). 1 mM EGTA+0.1 μM DTPA showed slightly increased oxidation, while 1 mM EGTA+0.1 mM DEF showed good protection (comparable with control). 1 mM EGTA+0.1 mM of both DEF and DTPA, however, provided less protection. Interestingly, higher concentrations (1 mM) of both DEF and DTPA failed to show improvement with either EDTA or EGTA.

### Example 6

### Effect of Chelators, Tween-80, Protein Concentration, Mannitol, Methionine, and/or Histidine on Metal and Ascorbate Induced Oxidation of Proteins

To examine the versatility of the oxidation protective compositions presented in the foregoing examples, two monoclonal antibodies and five other proteins and peptides were exposed to metals and ascorbate and additional combinations of chelators and scavengers of free radicals involving oxygen (referred to as reactive oxygen species or “ROS”). These experiments demonstrate that the oxidation protective and stabilizing compositions of the invention can be used with a variety of proteins which have a wide range of molecular weights, concentrations and biophysical and biochemical characteristics.

1. Effect on Oxidation of Monoclonal Antibodies

Two monoclonal antibodies (an anti-T lymphocyte antigen antibody and an anti-surface tumor antigen antibody) were examined in the presence of additional combinations of chelators and ROS scavengers.

The level of protection from oxidation was determined by SDS-PAGE using a gel concentration optimal for the molecular weight of the antibody. Gels were silver-stained, then scanned using a BIO-RAD GS-800 densitometer. The bands, representing an assortment of oxidative-related species (both aggregates and breakdown products) that were consistently observed, were detected and quantitated using the associated Quantity One software. Densitometric analysis provides an “adjusted volume”, i.e, the intensity of a band integrated over its volume, and adjusted for any staining background. Optimal protective mixtures should minimize the values of these adjusted volumes.

All antibody samples were treated with 4 mM Asc and metals (1 μM each Cu and Fe). The additional combinations tested included: no chelator, 100 μM DTPA and 20 μM DEF; 100 μM DTPA and 3% mannitol; 100 μM DTPA and 25 mM Methionine; 100 μM DTPA and 25 mM Histidine; 100 μM DTPA, 20 μM DEF and 25 mM Methionine; and 100 μM DTPA, 20 μM DEF and 3% methionine. The antibody protein solution contained 1 mg/mL protein in PBS, with either 0.01% Tween-80 or 2% glycerol. All samples were incubated at room temperature for at least 48 hrs, then stored at 4° C. The results (similar for both mAbs tested) are shown in Table 3.

TABLE 3

| Sample | Chelator | ROS Scavenger | Results |
|---|---|---|---|
| Control (mAb + | None | None | Typical pattern of |
| Tween-80 | | | heavy and light |
| with no | | | chain bands, plus |
| ascorbate or | | | low abundance of |
| metals added) | | | minor bands |
| Protein + Asc + | None | None | Substantial |
| Cu + Fe + | | | aggregation and |
| Tween-80 | | | breakdown |
| Protein + Asc + | DTPA + DEF | None | Significant decrease |
| Cu + Fe + | | | in aggregation and |
| Tween-80 | | | breakdown |
| Protein + Asc + | DTPA | Mannitol | Significant decrease |
| Cu + Fe + | | | in aggregation and |
| Tween-80 | | | breakdown |
| Protein + Asc + | DTPA | Methionine | Significant decrease |
| Cu + Fe + | | | in aggregation and |
| Tween-80 | | | breakdown |
| Protein + Asc + | DTPA | Histidine | Significant decrease |
| Cu + Fe + | | | in aggregation and |
| Tween-80 | | | breakdown |
| Protein + Asc + | DTPA + DEF | Methionine | Significant decrease |
| Cu + Fe + | | | in aggregation and |
| Tween-80 | | | breakdown |
| Protein + Asc + | DTPA + DEF | Mannitol | Significant decrease |
| Cu + Fe + | | | in aggregation and |
| Tween-80 | | | breakdown |
| Protein + Asc + | None | None | Substantial |
| Cu + Fe + | | | aggregation and |
| glycerol | | | breakdown |
| Protein + Asc + | DTPA + DEF | None | Significant decrease |
| Cu + Fe + | | | in aggregation and |
| glycerol | | | breakdown |

Values of relative protection against oxidation were also measured as follows: RP=100%−[(band intensity with protective compounds)÷(band intensity without protective compounds)]

The results of the relative oxidation protection levels for the monoclonal antibody protein samples are shown in Table 4. (A anti-T lymphocyte antigen antibody; B anti-surface tumor antigen antibody)

TABLE 4

| | Band MW | Rel. Protection Level |
|---|---|---|
| A | 164 kD | 90-98% |
| | 136 kD | 78-87% |
| | 90 kD | 25-63% |
| | 32 kD | 86-96% |
| B | 161 kD | 45-81% |
| | 133 kD | 50-62% |
| | 85 kD | 7-44% |
| | 62 kD | 18-40% |
| | 41 kD | 76-78% |
| | 26 kD | 89-99+% |

Results:

Without the presence of a combination of chelators or a combination of chelator and a ROS scavenger, both monoclonal antibody protein samples exhibited oxidation, as seen by new distinct bands visualized by silver-stained SDS-PAGE, as well as by an increase in intensity of bands known to contain oxidation, breakdown and aggregation-causing compounds such as Ascorbate, metals, Tween-80 and glycerol. For each of the conditions containing a combination of chelators or a combination of a chelator and a ROS scavenger, breakdown and aggregation of the protein samples was significantly diminished as evidenced by greatly reduced band intensities on the gels.

2. Protective Effect on Oxidation of IgG, BSA, hGH, PTH and ACTH

Human IgG, Bovine Serum Albumin (BSA), Human Growth Hormone, Parathyroid Hormone (PTH) and adrenocorticotropin hormoone (ACTH) were examined in the presence of additional combinations of chelators and ROS scavengers. The level of protection was determined by SD S-PAGE, using a gel concentration optimal for the molecular weight of the protein or peptide. Gels were silver-stained, then scanned using a BIO-RAD GS-800 densitometer. The bands, which represent an assortment of oxidative-related species (both aggregates and breakdown products) and were consistently observed, were detected and quantitated using the associated Quantity One software. Densitometric analysis provides an “adjusted volume”, i.e, the intensity of a band integrated over its volume, and adjusted for any staining background. Optimal protective mixtures should minimize the values of these adjusted volumes. The concentrations of the protein samples and the corresponding gels concentrations are summarized in Table 5.

TABLE 5

| Protein | Concentration | Volume | Notes |
|---|---|---|---|
| IgG | 1 mg/mL | 1 mL | 4-20% gels |
| BSA | 1 mg/mL | 500 uL | 4-20% gels |
| Human Growth Hormone | 50 ug/mL | 200 uL | 4-20% gels |
| Parathyroid Hormone | 0.5 mg/mL | 200 uL | 10-20% Tricine |
| ACTH | 1 mg/mL | 200 uL | 10-20% Tricine |

All protein samples were treated with 4 mM Asc and metals (1 μM each Cu and Fe). The additional combinations tested included: no chelator; 100 μM DTPA and 20 μM DEF; 100 μM DTPA and 3% mannitol; 100 μM DTPA and 25 mM Methionine; 100 μM DTPA and 25 mM Histidine; 100 μM DTPA, 20 μM DEF and 25 mM Methionine; and 100 μM DTPA, 20 μM DEF and 3% methionine. The antibody protein solution contained 1 mg/mL protein in PBS, with either 0.01% Tween-80 or 2% glycerol. All samples were incubated at room temperature for at least 48 hours, then stored at 4° C.

Results:

Upon exposure to metals and ascorbate, IgG exhibited an increase in higher molecular weight oxidation-induced aggregates, as well as breakdown products and species migrating between the heavy and light chains of the antibody oxidation, which was similar to the antibodies studied in the previous Examples. Introduction of combinations of oxidative protective compounds to the IgG samples reduced oxidative degradation.

Upon exposure to metal and ascorbate, BSA exhibited two major degradation products (intensity greater than that of the original “main band” (i.e., the band corresponding to the primary, most abundant protein species in the sample) on silver-stained SDS-PAGE. All combinations of DTPA and DEF, and chelators and ROS scavengers prevented the formation of these oxidative species (no detectable band).

Upon exposure to metals and ascorbate, human growth hormone showed a band having slightly smaller apparent molecular weight, possibly due to isomerization. This band was not detected in samples containing combinations of DTPA and DEF, and these chelators with and ROS scavengers.

Exposure to metals and ascorbate increased the number of aggregation species in PTH. Treatment with combinations of DTPA and DEF, and these chelators with ROS scavengers reduced the aggregation species.

Exposure to metals and ascorbate generated a number of aggregation species in ACTH. These species were diminished in samples containing combinations of DTPA and DEF, and these chelators with ROS scavengers.

Table 6 summarizes the results of the experiments with the aforementioned proteins and peptides. Overall, the inclusion of combinations of DTPA and DEF, and these chelators with ROS scavengers resulted in a quantifiable protective effect in a wide range of protein concentrations, protein sizes, and types of proteins (antibodies, hormones, etc).

TABLE 6

| | | MW of major | Additional or | |
|---|---|---|---|---|
| Protein/ | Theoretical | band by | Enhanced | Relative |
| Peptide | MW | SDS-PAGE | Species | Protection |
| IgG | 54/28 | 53.6 +/− 0.8; | 162, 135, | 10-72% |
| | | 28.5 +/− 0.4 | 89, 43, 40 kD | (dependent on |
| | | | | band) |
| BSA | 67 kD | 64 kD | 44, 33 kD | >98% |
| HGH | 22 kD | 24 kD | 22, 17 kD | >90% |
| PTH | 4 kD | 4 kD | 6 kD | 66-95% |
| ACTH | 3 kD | 4.5 kD | 7, 11 kD | 75-99% |

### Example 7

### Correlation with GPC-HPLC Methods of Studying Protein Oxidation GPC-HPLC

In addition to silver-stained SDS-PAGE, GPC-HPLC was used to investigate the occurrence of protein aggregation and changes in molecular weight and/or tertiary structure due to oxidation. The anti-T lymphocyte antigen antibody was used throughout this study. The focus of this study was on changes to the main monomer peak, i.e., the whole (unaffected) protein (typically eluting at 12 to 12.2 minutes), and development of aggregate peaks. Samples exposed to oxidative damage showed considerable changes in both main peak retention (decreased) and main peak shape (broadened). For protein samples where oxidative damage was extensive, Two additional peaks were seen with retention times of 9 to 9.5 minutes and 7.3 minutes (which corresponds to the column void volume). Ascorbate and metal chelators typically eluted with a retention time of 14 to 16 minutes, and the corresponding peaks were ignored.

Differences in protection between chelators were observed in samples exposed to copper and ascorbate. The DTPA treated sample showed a sharp monomer peak at 12.175 min, indicative of native antibody structure, while the main peak of the DEF sample was broadened and shifted (suggesting a change in antibody structure in comparison with whole native antibody) with a retention time of 11.5 min. EDTA, associated with the most oxidative damage observed by SDS-PAGE, also has a broad, shifted monomer peak and additional aggregate peaks.

In a similar set of protein samples treated with ascorbate and iron, the GPC-HPLC chromatograms parallel the SDS-PAGE results, i.e., DTPA provided less protection, which was seen in a broadened and shifted peak, while the DEF treated sample had a monomer peak closer to that of the native antibody. The chromatogram of the EDTA treated sample clearly showed the presence of oxidative damage.

Bioactivity/Binding Affinity

Four representative samples were selected to test for bioactivity. ELISA was used to determine bioactivity by measuring concentration of antibody required to titrate antigen to determine binding affinity. The results are shown in Table 7.

TABLE 7

| Sample | | | |
|---|---|---|---|
| Description | SDS-PAGE | GPC R.T. (min) | Bioactivity |
| DTPA, copper | Few bands | 12.175 | 85% |
| DTPA, iron | More bands (esp. | 11.937 | 63% |
| | breakdown) | | |
| EDTA, copper | Many bands, dark | 11.281 | 34% |
| DEF, copper | Most bands, very dark | 11.495 | 15% |

Results:

All three methods, i.e., SDS-PAGE, GPC and ELISA, consistently showed that oxidation causes damage to protein structure resulting in a considerable loss of activity, and that the addition of oxidation-protective excipients, such as DTPA, DEF, and combinations thereof, prevents loss of bioactivity due to the oxidative processes.

### Example 8

### Correlation with Photoxidation Methods of Studying Protein Oxidation

The bands observed in photooxidation of samples using SDS-PAGE align with the bands seen in chemically oxidized and real-time stability studies. The protein samples' molecular weights and elution profiles on gels suggest that the aggregates and breakdown products are product-related, e.g., oxidized linkages of heavy and light chains. This experiment focused on shorter timescale photooxidation experiments, run in parallel with chemical oxidation experiments. This allows a side-by-side comparison of species generated by the two types of oxidation processes.

Antibody containing protein samples (anti-immunoreceptor antibody and anti-T lymphocyte antigen antibody) were used as test proteins at concentrations of 1 mg/mL. Buffer conditions included PBS, as described previously and also DTPA (1 mM), Mannitol (10%), Methionine (15 mM) and Histidine (50 mM). DTPA (1 mM) and Mannitol (10%) were also combined. To generate chemical oxidation, samples were exposed to 4 mM ascorbate and 1 μM copper and iron. Samples were analyzed by silver-stained SDS-PAGE.

For both protein samples, development of distinct photooxidation species was observed after one day of 10× light treatment. Similar alignment of bands between anti-immunoreceptor antibody photooxidation species and chemical oxidation species was observed. However, in the anti-T lymphocyte antigen antibody, there were distinct differences between the photooxidation species and the chemical oxidation bands. This shows that formulations of the invention are effective at reducing oxidative species originating from different types of mechanisms.

Results:

The samples treated with 1 mM DTPA showed some improvement over unprotected samples. Samples treated with mannitol, DTPA/mannitol, and histidine also showed a protective effect. Methionine was particularly useful in reducing the formation of the photooxidative species visualized between the heavy and light chains on SDS-PAGE.

### CONCLUSIONS

The foregoing Examples demonstrate that oxidation causes considerable damage to proteins, particularly monoclonal antibodies, and that oxidative damage can be reduced by formulating proteins together with selected combinations of metal chelators, including DTPA, EGTA, and DEF, either alone or in combination with one or more ROS scavengers (such as mannitol, histidine and/or methionine). The foregoing examples further show that DTPA and DEF have an unexpected synergistic effect on reducing oxidation. In particular, a combination of at least 0.1 mM DTPA and 0.02 mM DEF, either with or without ROS scavangers, is effective as a universal additive to antibody and other protein formulations in protecting against oxidation. The foregoing examples still further show the destructive effect that the chelator EDTA has on proteins, and that the foregoing compositions can protect against oxidation or “rescue” proteins from this effect.

Equivalents

Those skilled in the art will recognize, or be able to ascertain using no more than routine experimentation, many equivalents to the specific embodiments of the invention described herein. Such equivalents are intended to be encompassed by the following claims. The entire contents of all references, patents and published patent applications cited throughout this application are hereby incorporated by reference.

1. A composition comprising a protein formulated with DTPA and another agent selected from the group consisting of DEF, mannitol, methionine, and histidine.

2. The composition of claim 1, comprising DEF.

3. The composition of claim 1, further comprising EGTA.

4. The composition of claim 1, wherein the concentration of DTPA is from about 1 μM to about 10 mM.

5. The composition of claim 2, wherein the concentration of DEF is from about 1 μM to about 5 mM.

6. The composition of claim 1, comprising mannitol at a concentration of about 0.01% to about 25%.

7. The composition of claim 1, comprising methionine at a concentration of about 10 μM to about 200 mM.

8. The composition of claim 1, comprising histidine at a concentration of about 100 μM to about 200 mM.

9. The composition of claim 1, further comprising an agent that inhibits protein aggregation.

10. The composition of claim 9, wherein the agent that inhibits protein aggregation is selected from the group consisting of polysorbate 80, polysorbate 20, glycerol, and a poloxamer polymer.

11. The composition of claim 10, wherein the agent that inhibits protein aggregation is polysorbate 80 or polysorbate 20 at a concentration of from about 0.001% to about 0.1%.

12. The composition of claim 1, further comprising a buffer that maintains the pH of the composition from about 5.0 to about 8.0.

13. The composition of claim 12, wherein the buffer is selected from the group consisting of phosphate, citrate, Tris, acetate, MES, succinic acid, PIPES, Bis-Tris, MOPS, ACES, BES, TES, HEPES, EPPS, ethylenediamine, phosphoric acid, and maleic acid.

14. The composition of claim 1, comprising mannitol, a polysorbate, Tris, and sodium chloride, wherein the protein is an antibody or fragment thereof.

15. The composition of claim 1, wherein the concentration of the protein is from about 1 μg/mL to about 500 mg/mL.

16. The composition of claim 1, wherein the protein is an antibody, or a fragment thereof.

17. The composition of claim 16, wherein the antibody is a monoclonal antibody, or a fragment thereof.

18. The composition of claim 16, wherein the antibody is a human antibody, or a fragment thereof.

19. The composition of claim 16, wherein the antibody is conjugated to an agent, selected from the group consisting of a toxin, a polymer, an imaging agent and a drug.

20. The composition of claim 1, wherein the protein is microencapsulated.

21. The composition of claim 1, wherein the composition is a pharmaceutical composition.

22. A composition comprising a protein formulated with EGTA and DEF.

23. A method for preparing a stabilized protein composition, comprising formulating a protein together with DTPA and another agent selected from the group consisting of DEF, mannitol, methionine, and histidine.

24. The method of claim 23, comprising DEF.

25. The method of claim 23, wherein the composition further comprises EGTA.

26. The method of claim 23, wherein the concentration of DTPA or EGTA is from about 1 μM to about 10 mM.

27. The method of claim 24, wherein the concentration of DEF is from about 1 μM to about 5 mM DEF.

28. The method of claim 23, wherein the oxidation protective compound is selected from the group consisting of about 0.01% to about 25% mannitol, about 10 μM to about 200 mM histidine, and about 10 μM to about 200 mM methionine.

29. The method of claim 23, further comprising adding an agent that inhibits protein aggregation to the composition.

30. The method of claim 23, further comprising adding a buffer that maintains the pH from about 5.0 to about 8.0 to the composition.

31. The method of claim 30, wherein the buffer is selected from the group consisting of about 5 mM to about 100 mM phosphate, citrate, Tris, acetate, MES, succinic acid, PIPES, Bis-Tris, MOPS, ACES, BES, TES, HEPES, EPPS, ethylenediamine, phosphoric acid, and maleic acid.

32. The method of calim 23, wherein the composition comprises mannitol, a polysorbate, Tris, and sodium chloride, wherein the protein is an antibody or a fragment thereof.

33. The method of claim 23, wherein the concentration of the protein is from about 1 μg/mL to about 500 mg/mL.

34. The method of claim 23, wherein the protein is an antibody, or a fragment thereof.

35. The method of claim 34, wherein the antibody is a human antibody, or a fragment thereof.

36. The method of claim 34, wherein the antibody is a monoclonal antibody, or a fragment thereof.

37. The method of claim 34, wherein the antibody is conjugated to an agent selected from a toxin, a polymer, an imaging agent or a drug.

38. The method of claim 23, wherein the protein is microencapsulated.

39. The method of claim 23, wherein the composition is a pharmaceutical composition.

40. The method of claim 23, wherein the protein is protected against oxidation.

41. A method for preparing a stabilized protein composition, comprising formulating a protein together with EGTA and DEF.

42. The method of claim 41, wherein the protein is protected against oxidation.
